(19) United States
(12) Patent Application Publication
(10) Pub. No.: US 2021/0321930 A1
(43) Pub. Date: Oct. 21, 2021

(54) ELECTRONIC DEVICE SET FOR BIOLOGICAL SUBJECT

(71) Applicant: BIOPRO SCIENTIFIC CO., LTD., Hsinchu City (TW)

(72) Inventors: HSING-HUA HO, HSINCHU CITY (TW); HSIN CHEN, HSINCHU CITY (TW); PIN CHANG, HSINCHU CITY (TW)

(21) Appl. No.: 17/142,880

(22) Filed: Jan. 6, 2021

Related U.S. Application Data
(60) Provisional application No. 63/010,664, filed on Apr. 15, 2020.

(51) Int. Cl. A61B 5/00; A61D 3/00

(52) U.S. Cl. CPC A61B 5/4064; A61D 3/00

(57) ABSTRACT

The application discloses an electronic device set including a modular appliance. The modular appliance includes a first power supplier, a processing device, and a connector. The power device includes a first power supply terminal, a second power supply terminal and at least one first signal terminal. The the processing device is configured to perform data acquisition and electrical stimulation on a biological subject via the at least one first signal terminal. The connector includes contacts coupled to the first electrode, the second electrode, the first power supply terminal, the second power supply terminal, and the at least one first signal terminal, respectively.

### CROSS-REFERENCE TO RELATED APPLICATIONS

This application claims the benefit of prior-filed provisional application with application No. 63/010,664, filed Apr. 15, 2020, which is incorporated by reference in its entirety.

### BACKGROUND

### Field of the Invention

The present disclosure relates to an electronic device set; in particular, to an electronic device set including a modular appliance, an adapter, a charger and a service station, wherein the modular appliance is able to be connected with the adapter, the charger or the service station depending on different application scenarios.

### Description of Related Art

In biological experiments, a modular appliance, such as a headstage, is connected with a biological subject under test through a mechanical part; for example, through an adapter mounted on laboratory rodent's head. Existing headstages have various disadvantages, for example, existing headstages need to be powered on and off manually. Therefore, how to mitigate inconvenience during biological experiments by improving flexibility of modular appliances is the problem to be solved in the instant case.

### BRIEF SUMMARY OF THE INVENTION

Some embodiments of the present disclosure provide an electronic device set including a modular appliance. The modular appliance includes a first power supplier, a processing device, and a connector. The power device includes a first power supply terminal, a second power supply terminal and at least one first signal terminal. The processing device is configured to perform data acquisition and electrical stimulation on a biological subject via the at least one first signal terminal. The connector includes contacts coupled to the first electrode, the second electrode, the first power supply terminal, the second power supply terminal, and the at least one first signal terminal, respectively.

The present device is able to improve the efficiency of utilizing the biological headstage device.

### BRIEF DESCRIPTION OF THE DRAWINGS

Aspects of the present disclosure are best understood from the following detailed description when read with the accompanying figures. It should be noted that, in accordance with the standard practice in the field, various features are not drawn to scale. In fact, the dimensions of the various features may be arbitrarily increased or reduced for clarity of discussion.

FIG. 1 is a schematic diagram of a modular appliance according to some embodiments of the present disclosure.

FIG. 2 is a schematic diagram of an electronic device set according to some embodiments of the present disclosure.

FIG. 3 is a schematic diagram of an electronic device set according to various embodiments of the present disclosure.

FIG. 4 is a schematic diagram of an electronic device set according to alternative embodiments of the present disclosure.

### DETAILED DESCRIPTION

The following disclosure provides many different embodiments, or examples, for implementing different features of the provided subject matter. Specific examples of elements and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Further, spatially relative terms, such as “beneath,” “below,” “lower,” “above,” “upper”, “on” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

As used herein, the terms such as “first”, “second” and “third” describe various elements, components, regions, layers and/or sections, these elements, components, regions, layers and/or sections should not be limited by these terms. These terms may be only used to distinguish one element, component, region, layer or section from another. The terms such as “first”, “second”, and “third” when used herein do not imply a sequence or order unless clearly indicated by the context.

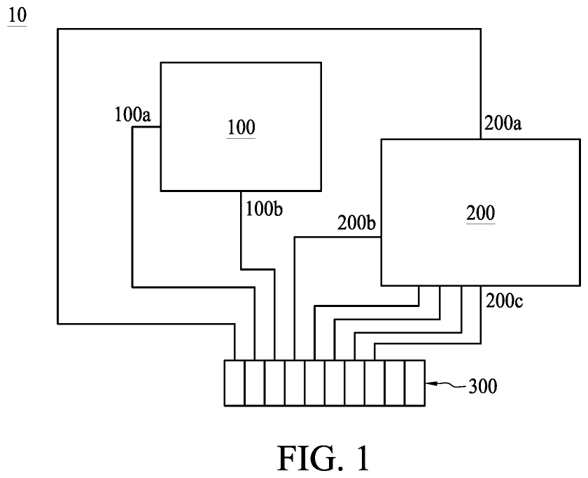

Reference is made to FIG. 1. FIG. 1 is a schematic diagram of a modular appliance 10 according to some embodiments of the present disclosure. The modular appliance 10 may be a headstage for biological experiments. For example, when operated, the modular appliance 10 may be connected with a biological subject under test through a mechanical part; for example, through an adapter mounted on laboratory rodent's head, such as a rat's or a mouse's head. However, this is not a limitation of the present disclosure. The modular appliance 10 may also be applied to any other applications when appropriated.

In FIG. 1, the modular appliance 10 includes a power reservoir 100, a processing device 200, and a connector 300. The power reservoir 100 is coupled to the processing device 200, and the power reservoir 100 and the processing device 200 are coupled to the connector 300.

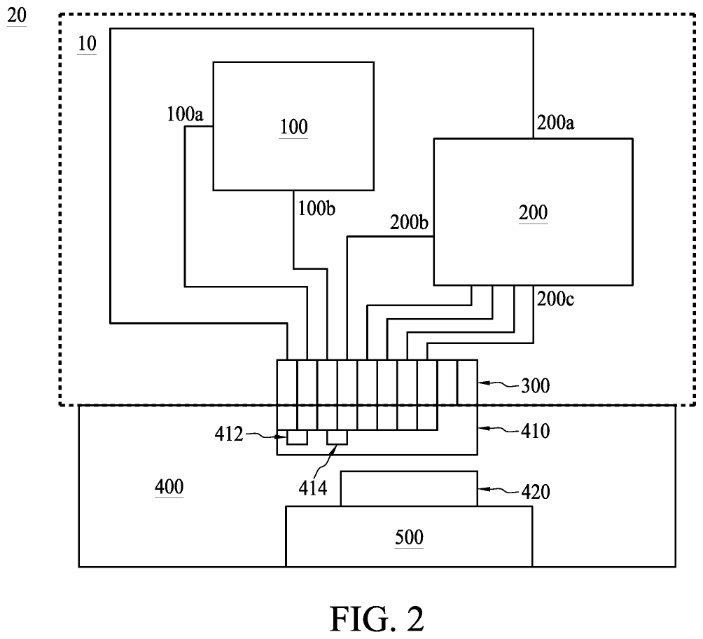

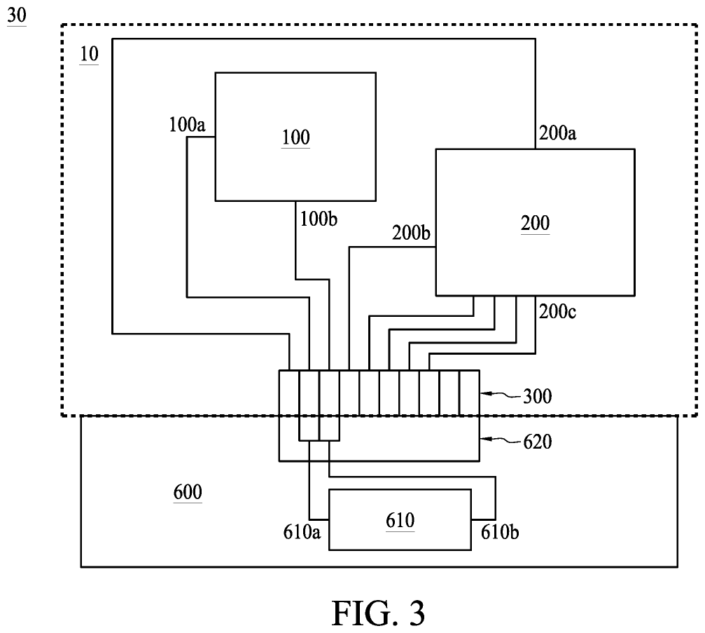

The power reservoir 100 is configured to provide power. For example, the power reservoir 100 provides power to the processing device 200 when the modular appliance 10 is coupled to an adaptor as shown in FIG. 2, which is described in the following paragraphs. The processing device 200 performs various functions such as sense, excite, trigger, stimulate, process, observe, and/or data acquisition. In some embodiments, the processing device 200 further configured to perform laboratory automation, test and measurement, bench top experiment, and field observation. The power reservoir 100 may be a battery including an electrode 100a and an electrode 100b, and the processing device 200 includes a power supply terminal 200a, a power supply terminal 200b, and at least one signal terminal 200c. In some embodiments as shown in FIG. 3, the power reservoir 100 is a rechargeable battery, which is described in the following paragraphs.

Please note that four signal terminals 200c depicted in FIG. 1 are for illustrative purpose only. The number of the signal terminals 200c is not a limitation. For example, the processing device transmits a transmitting signal through two signal terminals 200c to the connector 300, and receives receiving signal by the other two signal terminals 200c from the connector 300. When another device is coupled to the processing device 200 through the connector 300, the processing device 200 is able to perform signal processing and communicate with the device through the signal terminals 200c and the connector 300.

As shown in FIG. 1, the connector 300 is in connection with connection paths sourced from the terminals and electrodes of the power reservoir 100 and the processing device 200. In particular, the connector 300 has a plurality of contacts each connected to one of the connection paths sourced from the terminals and electrodes of the power reservoir 100 and the processing device 200. In that way, the connector 300 serves as an interface of the modular appliance 10. That is, many of the terminals and electrodes of the power reservoir 100 and the processing device 200 can be accessed through contacts of the connecter 300. The connector 300 may be connected with a corresponding connector through appropriate mechanical mechanisms. So that the connecter 300 may be easily plugged in and out from another device having the corresponding connector.

In some embodiments, the electrode 100b is coupled to the power supply terminal 200b through an internal connection path. The internal connection path is reserved between the electrode 100b and the power supply terminal 200b within the modular appliance 10, for example, within a housing of the modular appliance 10. In other words, there is no need and no way to manually arrange a connection path outside of the modular appliance 10 through the connector 300 in order to couple the electrode 100b to the power supply terminal 200b. In some other embodiments, the electrode 100b is further coupled to the ground of the modular appliance 10.

References are made to FIG. 2 to FIG. 4, which show electronic device sets 20, 30, and 40 including modular appliance 10 connected to an adapter 400, a charger 600, and a service station 700 respectively. To facilitate understanding, like elements in FIG. 2 to FIG. 4 are designated with the same numerals as shown in FIG. 1.

For illustration in FIG. 2, the modular appliance 10 is connected to and fixed on a connector 410 of the adapter 400 through appropriate mechanical mechanisms, and the electronic device set 20 as a whole is coupled to the subject 500 via a biological connection 420 of the adapter 400. In some embodiments, the subject 500 is a mouse in the biological laboratory, and the modular appliance 10 is mounted on the mouse's head by connecting the connector 410 of the adapter 400 for investigating activities of the mouse. For example, when the mouse's brain is excited by an external stimulus (such as a whiff of food) generated from the processing device 200, the brain generates a corresponding activity (such as a desire to eat) with a responding signal in an electrical form. The processing device 200 detects and analyzes the responding signal through the adapter 400. On the other hand, the processing device 200 may send a specific stimulus to the mouse's brain when a predefined pattern from the mouse's brain is detected.

In some embodiments, the biological connection 420 is permanently fixed on the subject 500. For example, the subject 500 is a mouse, and the biological connection 410 is surgically attached on the mouse's head. The biological connection 410 brings the contacts of the connector 410 to electrodes which are implanted into the mouse's head. When the modular appliance 10 is not in use, the modular appliance 10 is detached from the adaptor 400 by disconnecting from the connector 410, and the connector 410 still stays with the mouse. In various embodiments, the biological connection 420 is omitted in the adapter 400. The contacts of the connector 410 are surgically attached to the electrodes in the mouse's head.

When the modular appliance 10 is mounted on the adapter 400, a connection path 412 in the adapter 400 provides a circuit connection between the electrode 100a and the power supply terminal 200a, and a connection path 414 in the adapter 400 provides a circuit connection between the electrode 100b and the power supply terminal 200b. Therefore, the power reservoir 100 can deliver necessary electrical power to the processing device 200. For example, the connection path 412 and the connection path 414 are implemented by conducting traces connecting the two contacts of the connector 410. As a result, the modular appliance 10 is able to be automatically powered on by connecting to the adapter 400. In some embodiments, the connector 410 further couples the electrode 100b to the ground of the subject 500.

For illustration in FIG. 3, the electronic device set 30 includes the modular appliance 10 mounted on the charger 600. In some embodiments, the power reservoir 100 is a rechargeable battery. When the power reservoir 100 run out of power, the modular appliance 10 may be mounted on the charger 600 to charge the power reservoir 100. The charger 600 includes a power supplier 610 and a connector 620. The modular appliance 10 is connected to the charger 600 via the connector 610. More specifically, the connector 620 couples to the contacts of the connector 300 sourced from the electrode 100a and the electrode 100b to an electrode 610a and an electrode 610b of the power supplier 610, respectively. Therefore, a close loop between the power reservoir 100 and the power supplier 610 is formed, and the power supplier 610 can charge the power reservoir 100 through the close loop.

In addition, the connector 620 is configured to float the contacts of the connector 300 sourced from the power supply terminal 200a, the power supply terminal 200b, and the at least one signal terminal 200c. Therefore, when the modular appliance 10 is mounted on the charger 600, the processing device 200 keeps unpowered.

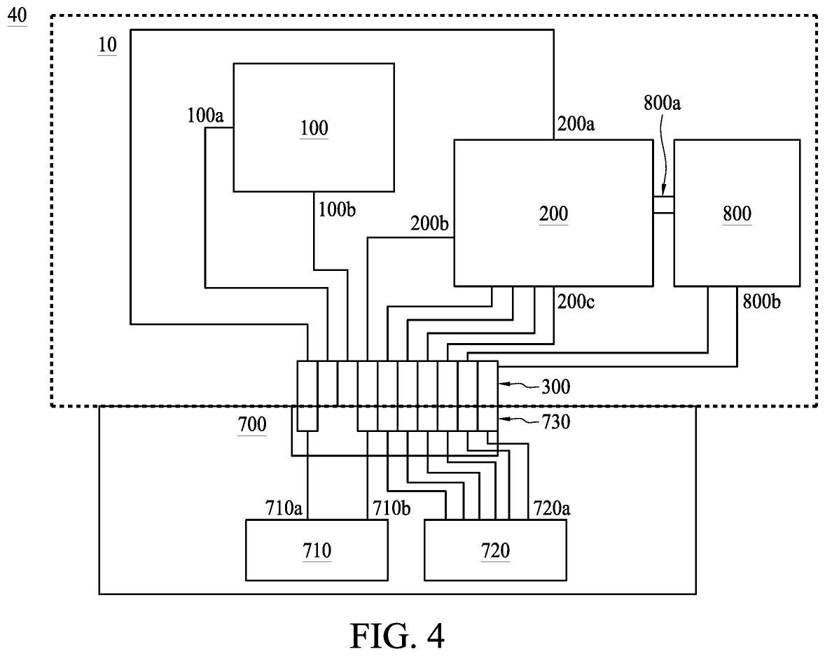

In some operations, the modular appliance 10 is connected to the service station 700 as shown in FIG. 4. The service station 700 is configured to communicate with the modular appliance 10 by data exchange. The data may be the result of signal processing performed by the processing device 200 while the modular appliance 10 is connected to the subject 500 through the connector 410, and the data is stored in a storage device (such as a memory, not shown in the drawings) of the modular appliance 10. For example, when the modular appliance 10 receives a signal which is induced by a twitching of the mouse (i.e., the subject 500), the processing device 200 configures the signal and transforms the signal as a data stored in the storage device, in which the data may include the information of the amplitude, the frequency, and the duration of the signal.

In some embodiments, the service station 700 is configured to download the data from the modular appliance 10. After a period of investigating the subject 500 by the modular appliance 10, the processing device 200 may collect enough data for the following biological experiments. Then, the modular appliance 10 is removed from the adapter 400 and mounted on the service station 700 for the following biological data analysis. For example, the service station 700 may computes an amount of data which has the amplitude higher than a threshold.

Moreover, after a certain period of performing the operation, the experiment environment (for example, the temperature and the pressure) or needs (for example, a novel characteristic in neural signal that should trigger an electrical stimulation) may change. Therefore, the software or firmware of the processing device 200 has to be modified to meet the current experiment environment or needs. The service station 700 is configured to calibrate/update/adjust/fine-tune the software or firmware of the processing device 200 in response to the change. After calibrating/updating/adjusting/fine-tuning the processing device 200, the modular appliance 10 may be capable of improved performance.

For example, when the subject 500 is changed to another species or when the modular appliance 10 needs new functions, the modular appliance 10 is removed from the adapter 400 and mounted on the service station 700. The service station 700 is configured to update the software or firmware of the processing device 200 in order to make the modular appliance 10 competent for the newly needs.

The service station 700 includes a power supplier 710, a processor 720, and a connector 730. The service station 700 is coupled to the modular appliance 10 through the connector 730. When the service station 700 performs the abovementioned operations, the power supplier 710 is configured to provide the power for the electronic device set 40, and the power reservoir 100 is idle. The connector 730 includes contacts coupled to the connector 300. The connector 730 is configured to couple an electrode 710a and an electrode 710b of the power supplier 710 to the contacts of the connector 300 sourced from the power supply terminal 200a and the power supply terminal 200b, respectively. Thus, a close loop for the power delivery is formed, in which the close loop starts from the electrode 710a to the electrode 710b via the power supply terminal 200a, the processing device 200, the power supply terminal 200b, the connector 300, and the connector 730. In other words, when the modular appliance 10 is connected to the service station 700, the modular appliance 10 is automatically powered on by the power supplier 710, and the power reservoir 100 keeps unloaded.

The connector 730 is further configured to couple the processer 720 to the connector 300. The processer 720 includes at least one signal terminal 720a coupled to the connector 730. The processer 720 is configured to download the data, calibrate the processing device 200, and update the processing device 200 through the at least one signal terminal 720a. For example, the processor 720 sends an initial signal through the at least one signal terminal 720a to the processing device 200, and informs the processing device 200 to start a download operation. Then, the processor 720 acquires, through the at least on signal terminal 720a, the data in the processing device 200 and stores the acquired data in other storage device in the service station 700 for further processing/analysis required by the experiments.

In some embodiments, the modular appliance 10 further includes an auxiliary device 800. The auxiliary device 800 is coupled to the processing device 200 and the connector 300. The auxiliary device 800 includes at least one signal terminal 800a coupled to the processing device 200 and at least one signal terminal 800b coupled to the connector 300.

The auxiliary device 800 is configured to provide assistance to the processing device 200 to perform the signal process. In other embodiments, the auxiliary device 800 is configured to provide assistance to the processor 720 of the service station 700 to perform calibration, updating software or firmware, and downloading data.

In some embodiments, the auxiliary device 800 is a memory device. For example, the auxiliary device 800 is an SD memory module. In some operations, the processing device 200 generates large amount data (for example, eight hours of recording data) and writes the data to the auxiliary device 800 through the at least one terminal 800a during the operations. However, when the processor 720 needs to access the large amount data, the transmission on the at least one terminal 800a may be too slow to slow down the operation of the processor 720. The modular appliance 10 may not have enough power to support high speed transmission between the processing device 200 and auxiliary device 800. Therefore, in this embodiments, the at least one terminal 800b may be an USB protocol which is able to support high speed data transmission and powered by the power supplier 710. The processor 720 is able to quickly access the data store in the auxiliary device 800 through the at least one terminal 800b. Alternatively stated, the processor 720 is able to communicate with the auxiliary device 800 without going through the processing device 200. For example, during the data acquisition, the at least one terminal 800b provides a high throughput data download path, and the processor 720 can retrieve the data in a short time.

The present disclosure provides the modular appliance 10 that is automatically powered on when connecting to the adapter 400 or the service station 700; and, on the other hand, the modular appliance 10 is automatically powered off when disconnecting from the adapter 400 or the service station 700. In addition, the modular appliance 10 is automatically charged when connecting to the charger 600. Because of automatically powering on and off, the convenience of operating the modular appliance 10 is improved. The need for a switch on the modular appliance 10 is eliminated. A manual switch can be relatively bulky on a miniaturized device. The time cost during the operations is reduced. Accordingly, the efficiency is improved as the time cost is reduced.

The foregoing outlines features of several embodiments so that those skilled in the art may better understand various aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent embodiments still fall within the spirit and scope of the present disclosure, and they may make various changes, substitutions, and alterations thereto without departing from the spirit and scope of the present disclosure.

What is claimed is:

1. An electronic device set, comprising:
a modular appliance including:
a power reservoir including a first electrode and a second electrode;
a processing device including a first power supply terminal, a second power supply terminal and at least one first signal terminal, and the processing device is configured to perform data acquisition and electrical stimulation on a biological subject via the at least one first signal terminal; and
a connector including a plurality of contacts coupled to the first electrode, the second electrode, the first power supply terminal, the second power supply terminal, and the at least one first signal terminal respectively.

2. The electronic device set of claim I, further comprising:
an adapter including:
a connector for coupling the adapter to the modular appliance, the connector of the adapter including a plurality of contacts including a first terminal, a second terminal, and at least one second signal terminal; and
a biological connection for coupling the adapter to the biological subject, the biological connection including at least one third signal terminal,
wherein
the second terminal is coupled to the first terminal through a connection path, and
when the connector of the modular appliance is connected to the connector of the adapter through a mechanical mechanism, the first power supply terminal is coupled to the first terminal, the first electrode is coupled to the second terminal, the at least one first signal terminal is coupled to the least one second signal terminal, so that the first electrode is coupled to the first power supply terminal.

3. The electronic device set of claim 1, wherein the power reservoir is a battery.

4. The electronic device set of claim 2, wherein when the connector of the modular appliance is connected to the connector of the adapter, the modular appliance transmits a stimulus signal to the biological subject and performs data acquisition in response to the stimulus signal through the adaptor.

5. The electronic device set of claim 1, further comprising:
a charger, including:
a first power supplier including a third electrode and a fourth electrode; and
a connector for coupling the charger to the modular appliance, the connector of the charger including a plurality of contacts coupled to the third electrode and the fourth electrode respectively,
wherein when the connector of the modular appliance is connected to the connector of the charger through a mechanical mechanism, the first electrode and the second electrode of the modular appliance is coupled to the third electrode and the fourth electrode of the charger respectively.

6. The device of claim 5, wherein when the connector of the modular appliance is connected to the connector of the charger, the first power supply terminal of the modular appliance is decoupled from the power reservoir and the first power supplier.

7. The device of claim 1, further comprising:
a service station including:
a second power supplier including a fifth electrode and a sixth electrode;
a processor including at least one fourth signal terminal; and
a connector for coupling the service station to the modular appliance, the connector of the service station including a plurality of contacts coupled to the fifth electrode, the sixth electrode and the at least one fourth signal terminal,
wherein when the connector of the modular appliance is connected to the connector of the service station through a mechanical mechanism, the first power supply terminal, the second electrode, the at least one first signal terminal is coupled to the fifth electrode, the sixth electrode and the at least one fourth signal terminal respectively.

8. The device of claim 7, wherein when the connector of the modular appliance is connected to the connector of the service station, the service station calibrates the modular appliance by using the processor.

9. The device of claim 7, wherein when the connector of the modular appliance is connected to the connector of the service station, the service station updates software or firmware of the processing device by using the processor.

10. The device of claim 7, wherein when the connector of the modular appliance is connected to the connector of the service station, the service station downloads data stored in the processing device to the service station by using the processor.
